(19) United States
(12) Patent Application Publication
(10) Pub. No.: US 2025/0325743 A1
(43) Pub. Date: Oct. 23, 2025

(54) DRIVE COUPLING AND SENSING SYSTEMS, AND METHODS FOR FLUID DELIVERY PUMPS

(71) Applicant: Beta Bionics, Inc., Irvine, CA (US)

(72) Inventors: Ryan Schoonmaker, Escondido, CA (US); Patrick John Castanaga, Escondido, CA (US); Martin Iwanowicz, San Diego, CA (US); Sydney Fairchild, San Diego, CA (US)

(73) Assignee: Beta Bionics, Inc., Irvine, CA (US)

(21) Appl. No.: 19/183,101

(22) Filed: Apr. 18, 2025

Related U.S. Application Data
(60) Provisional application No. 63/636,176, filed on Apr. 19, 2024.

(51) Int. Cl. A61M 5/142; A61M 5/145; A61M 5/172

(52) U.S. Cl. CPC A61M 5/14248; A61M 5/14236; A61M 5/1452; A61M 5/1723; A61M 2005/14268; A61M 2005/14506; A61M 2202/07; A61M 2230/201

(57) ABSTRACT

A pump device for pushing a metered quantity of a stored volume of a fluid medicament into a patient includes a drive motor including a rotational output rotating about a motor axis when energized. A piston is receivable within a reservoir including a reservoir axis and having an interior space in which the volume of the medicament is receivable and an outlet through which the metered quantity is expellable. The piston is movable within the reservoir along the reservoir axis so as to displace the metered quantity when moved in a direction of the outlet, The reservoir is located laterally adjacent to the drive motor, the reservoir axis and the motor axis running codirectionally. A leadscrew which is rendered non-rotatable and is affixed to the piston. A leadscrew advancement element is threadably engageable with the leadscrew and substantially fixed with respect to movement along the reservoir axis, rotation of rotation of the leadscrew advancement element imparting movement of said piston along said reservoir axis. The pump device further includes a mechanism operable to impart rotation to the leadscrew advancement element created by the drive motor.

### CROSS-REFERENCE TO RELATED APPLICATIONS

The present application claims priority to U.S. Provisional Application No. 63/636,176, filed Apr. 19, 2024, the contents of which are incorporated herein by reference in their entirety.

### BACKGROUND OF THE INVENTION

### Technical Field

This disclosure relates to fluid delivery systems, including medical devices that provide glucose control therapy to a subject, glucose level control systems, and ambulatory medicament pumps that deliver medicaments to subjects to control glucose levels in subjects, as any manner of pumping devices and/or patch pumps and other related fluid control mechanisms driving insulin and other therapies to patients, including bi-hormonal or glucagon and related medicaments delivery among other things.

### Description of Related Art

Fluid delivery devices are known for treating type-1 diabetes in particular. U.S. Pat. Nos. 11,957,876; 11,571,570; 11,610,661; 11,610,662; 11,612,719, 11,633,635; 11,679,701; 11,688,501; 11,698,785; 11,716,598; 11,794,947 (assigned to the instant assignee) define the state of art which has evolved from lead screw advancement to drive fluid delivery as shown also in U.S. Pat. Nos. 11,229,741; 10,420,883 and 9,420,950 for advancing lead screws and plungers inside of pumps, including patch pumps. Prior to the advent of the instant systems, this was one way that fluid was pushed, all of the way back to U.S. Pat. No. 7,008,403, wherein the Ideal Gas Law was leveraged to utilize gas pressurized medicament movement along with non-fluid contact sensors.

With the current state of the art, including U.S. Pat. Nos. 11,744,947; 11,925,788 and 11,941,788 (as explained in detail below) achievements include sustained delivery, pump driven medicament injection devices which generally include a delivery cannula mounted in a subcutaneous manner through the skin of the subject at an infusion site. The pump expels medicine from a reservoir and delivers it to the subject via the cannula. The injection device typically includes a channel that transmits a medicament from an inlet port to the delivery cannula which results in delivery to the subcutaneous tissue layer where the delivery cannula terminates. Some infusion devices are configured to deliver one medicament to a subject while others are configured to deliver multiple medicaments to a subject. The medicament and/or supplies (infusion sets, analyte sensors, transmitters, and/or other components), must be monitored and periodically replaced, which requires that the subject keep track of the amount of medicament and/or supplies left. Failure to maintain an adequate supply of medicament and other supplies can disrupt treatment. However, the vast improvements in pushing fluids, standing at the cutting edge, still require more modifications to adequately treat patients and keep the tracking and communicating functions in lock-step with such mechanical and fluid-dynamics technologies.

By using a clutch mechanism, the engagement between the leadscrew and the nut occurs at assembly, and thus no rotation is needed for the nut to engage the leadscrew by operation of the device. This reduces the number of fluid path prime pulses to prime the pump and assures a full and proper priming of the fluid path before placement on the body. The clutch mechanism also enables the changing of thread pitch for other drug applications without a need to redesign the tilt nut used in fluid driving mechanisms in other existing pumps, as expressly labeled as desiderata by industry, including over the INSULET OMNIPOD, a brand of device (see US Patent offer one set of such systems.

Briefly stated, the present inventors have inter alia created a system that supplants the strict need for any of prior suggested ‘clutch-types of mechanisms’ to drive fluid from pumps for delivering medicaments to patients, with any and all manners of pumps, used for example, to 10,420,883, Column 2, line 2).

Such systems, while highly controverted and the subject of ongoing patent litigations, only serve to underscore a need for improvements by the antiquated and longstanding mechanical challenges called out by these needs. Accordingly, even in germinal stages, such modifications are embraced by and soon to become new standards of care in the industry, once again qualifying as progress in sciences and the useful arts, it is respectfully proposed and offered for consideration herein systems addressing such drawbacks of the prior art.

Driving the prior art was the fact that by using a clutch mechanism, the engagement between the leadscrew and the nut occurs at assembly, and thus no rotation is needed for the nut to engage the leadscrew by operation of the device. This reduces the number of fluid path prime pulses to prime the pump and assures a full and proper priming of the fluid
path before placement on the body. The clutch mechanism also enabled the changing of thread pitch for other drug applications without a need to redesign the tilt nut used in fluid driving mechanisms in other existing pumps.

Improvement in this area underline and highlight the needs for better ways to push fluids, as the balance of the state of the art advances at rapid-fire pace. It is therefore proposed that such matters require full attention and as proposed constitute progress in science and the useful arts worthy of United States Letters Patents.

### SUMMARY OF THE INVENTION

The systems, methods, and devices of this disclosure each have several innovative aspects, no single one of which is solely responsible for all the desirable attributes disclosed herein. The instant approach seeks to evolve past the mechanically challenged “clutching” which holds back much of the field. Details of one or more implementations of the subject matter described in this specification are set forth in the accompanying drawings and the description below.

Ambulatory medical devices, ambulatory medicament pumps and/or any manner of patch pumps require improved fluid drive mechanisms, as illustrated herein and claimed below.

According to features of the present invention there are provided novel enhanced drive coupling and sensing mechanisms systems and methodologies, comprising, in combination: at least a one way drive coupling and fill detection apparatus using contact switch actuation and travel to interface with power management for both disposable and durable options in pumps for use with delivery of therapy for diabetes.

It is an object of the invention to provide novel infusion pumps which overcome the drawbacks of the prior art, and which are advantageously provided in a form which is relatively compact, light-weight and easy to use in the context of portability, each conveniently provided in a form of a patch pump wearable by a user. Such patch pumps represent improvements in certain clinical settings, but have enumerated strong needs to improve fluid driving mechanisms from reservoirs through transcutaneous access tools. In this regard, the embodiment described herein deliver therapy for diabetes. This extends from conventional systems to patch pumps and later developed technologies. To make these disclosures, both the evolution of said pumps, and the instant improvements are explained herein, to connect the improvements to the historical developments. It is hereby earnestly solicited that such step changes are patentable as new, novel and non-obvious.

With the embodiments of the invention, from priming methods through all types of fluid
delivery, which advantageously comprise both disposable and durable systems, power management is improved, enabling both rechargeable non-rechargeable paradigms.

Some embodiments described herein pertain to medicament infusion systems for one or more medicaments and the components of such systems (e.g., infusion pumps, medicament cartridges, cartridge connectors, lumen assemblies, infusion connectors, infusion sets, etc.).

Some embodiments pertain to methods of manufacturing infusion systems and components thereof.

Other features are directed to methods of using any of the foregoing systems or components for infusing one or more medicaments (e.g., pharmaceutical, hormone, etc.) to a subject recipient. As an exemplary illustration, an infusion system may include an infusion pump, which can include one or more medicament cartridges or can have an integrated reservoir of medicament.

In is noted that an infusion system may include medicament cartridges and cartridge connectors, but not a pump. An infusion system may include cartridge connectors and an infusion pump, but not medicament cartridges. An infusion system may include infusion connectors, a lumen assembly, cartridge connectors, an infusion pump, but not medicament cartridges or an infusion set.

A glucose level control system can operate in conjunction with an infusion system to infuse one or more medicaments, including at least one glucose level control agent, into a subject. An infusion system may include a glucose sensor interface that can receive glucose level signals from a glucose sensor operatively connected to a subject. The glucose level signals may be received via a wireless link established between the glucose sensor and the infusion system. The infusion system may have a controller that controls the infusion of the medicament from the medicament cartridge to the subject based at least in part on the received glucose level signals. The controller of the infusion system may analyze the glucose level data associated with the received glucose level signals to identify glucose level artifacts based on the temporal behavior or the magnitude of the glucose level and determine the medicament dose and delivery time based at least in part on the identified glucose level artifacts.

Any feature, structure, component, material, step, or method that is described and/or illustrated in any embodiment in this specification can be used with or instead of any feature, structure, component, material, step, or method that is described and/or illustrated in any other embodiment in this specification. Additionally, any feature, structure, component, material, step, or method that is described and/or illustrated in one embodiment may be absent from another embodiment.

Further, certain embodiments disclosed herein relate to a glucose level control system that is capable of supporting different operating modes associated with different adaptation ranges used to generate dose control signals for delivering medicament to a subject.

The different adaptation ranges may be optionally associated with a value or a change in value of one or more control parameters used by a control algorithm that controls the administering of medicament to a subject. In some non-limiting examples, the control parameter may be associated with the quantity of medicament, a delivery rate of medicament, a step-size or graduation used to modify the quantity of medicament between administrations of the medicament, a timing of supplying medicament to the subject, a glucose absorption rate, a time until the concentration of insulin in blood plasma for a subject reaches half of the maximum concentration, a time until the concentration of insulin in blood plasma for a subject reaches a maximum concentration, or any other control parameter that can impact a timing or quantity of medicament (e.g., insulin or counter-regulatory agent) supplied or administered to a subject.

Advantageously, in certain embodiments, supporting different operating modes enables a user (e.g., a healthcare provider, parent, guardian, the subject receiving treatment, etc.) to modify the operating mode of an ambulatory medicament device. In some aspects, the operating mode may be modified automatically. Moreover, modifying the operating mode enables different dosing modes to be supported. Advantageously, supporting different dosing modes enables an ambulatory medicament device to be used by different types of subjects, and/or a subject under different conditions (e.g., when exercising, before, during, or after puberty, under different health conditions, etc.).

Detailed descriptions and examples of systems and methods according to one or more illustrative embodiments of the present disclosure may be found, at least, throughout this disclosure. Furthermore, components and functionality for supporting high dose mode or multiple operating modes may be configured and/or incorporated into the ambulatory medical device.

Some patients manage their diabetes by injecting insulin, which may be referred to as injection therapy. Other patients use medicament pumps to help manage their diabetes.

These medicament pumps may be controlled manually or may be closed using an autonomous system. For example, a glucose level control system may operate in a closed loop mode that enables the glucose level control system to automatically determine insulin dosing using a control algorithm and one or more sensor signals received at a sensor interface from one or more sensors. These sensors may include continuous glucose monitoring (CGM) sensors operatively coupled to a subject. The CGM sensors may provide measurements of glucose levels of the subject to the glucose level control system, which may autonomously determine the insulin dose using the measurements.

Glucose level control systems that autonomously determine a quantity of medicament (e.g., insulin or counter-regulator agent, such as Glucagon) to supply to a patient are becoming more common. The use of glucose level control systems and medicament pumps free
the patient from the inconvenience of injection therapy. Furthermore, it is recognized that automated closed-loop control algorithms are generally more accurate and provide better disease maintenance than other diabetes management options. For example, using test strips to measure glucose levels and performing injection therapy based on the test strips tends to be less accurate as it may not account for previously injected insulin or recent food consumption.

Nevertheless, some patients are hesitant to give complete control of their diabetes management to an automated glucose level control system. For example, a patient who is used to manually controlling his or her insulin intake or manually managing his or her disease may not feel comfortable partially or fully giving up control of insulin or management of diabetes to an automated glucose level control system. Moreover, regardless of whether the administration of medicament is optimal for a patient (in general or at a particular point in time), a patient may subjectively not feel that the supplied medicament is correct in quantity and/or timing. However, in some aspects, there may be objective reasons that the patient believes the quantity of medicament supplied is not appropriate or optimal. For example, a patient may be aware of or anticipating unusual meal or exercise activity. Although the glucose level control system of the present disclosure can account for unusual activity, some glucose level control systems may not and/or the patient may not feel comfortable relying on the glucose level control system to account for the unusual activity. Thus, for the above reasons, patients (also referred to as “subjects”) or users (e.g., parents, guardians, etc.) may desire to operate the glucose level control system manually or to manually adjust automatic determinations by the automated glucose level control system.

Closed loop or hybrid closed loop automated insulin delivery systems may advantageously use basal rates as a starting point from which insulin delivery is modulated. In some systems these may be entered manually or set autonomously. Embodiments of the present disclosure describe a system and method for adjusting these basal rates in order to optimize glucose control. Further, embodiments of the present disclosure describe a system and method for adjusting insulin (or other medicament) doses or dose rates (e.g., meal dose rates, corrective dose rates, etc.).

In certain embodiments, a user can enter or adjust a basal rate or basal rate segment (e.g., daytime or nighttime basal rate). The adjusted rate may be used by a control algorithm to modulate delivery of insulin, or other medicament. The ability to have a user manually adjust the basal rate may be particularly useful for experienced users who want more control of their diabetes management.

### Glucose Level Control System Overview

A glucose level control system (GLCS) is used to control glucose level in a subject. In some aspects, glucose level may comprise blood glucose level, or glucose level in other parts or fluids of the subject's body. In some examples, glucose level may comprise a physiological glucose level of the subject that can be a concentration of glucose in subject's blood or an interstitial fluid in part of the subject's body (e.g., expressed in milligram per deciliter (mg/dl)). Glucose level control systems (GLCSes) or glucose control systems, which can be referred to herein as glucose level systems or glucose control systems, can include a controller configured to generate dose control signals for one or more glucose control agents that can be infused into the subject. Glucose control agents can be delivered to a subject via subcutaneous injection, via intravenous injection, or via another suitable delivery method. In the case of glucose control therapy via an ambulatory medicament pump, subcutaneous injection is most common. Glucose control agents may include regulatory agents that tend to decrease a glucose level (e.g., blood glucose level) of the subject, such as insulin and insulin analogs, and counter-regulatory agents that tend to increase a glucose level of the subject, such as glucagon or dextrose. A glucose level control system configured to be used with two or more glucose control agents can generate a dose control signal for each of the agents. In some embodiments, a glucose level control system can generate a dose control signal for an agent even though the agent may not be available for dosing via a medicament pump connected to the subject.

In some embodiments, a GLCS may include or can be connected to an ambulatory medicament pump (AMP). An ambulatory medicament pump is a type of ambulatory medical device (“AMD”), which is sometimes referred to herein as an ambulatory device, an ambulatory medicament device, or a mobile ambulatory device. In various implementations, ambulatory medical devices include ambulatory medicament pumps and other devices configured to be carried by a subject and to deliver therapy to the subject. Multiple AMDs are described herein. It should be understood that one or more of the embodiments described herein with respect to one AMD may be applicable to one or more of the other AMDs described herein. In some aspects, a GLCS can include a therapy administration system and an AMD that is in communication with the therapy administration system. In some aspects, the AMD may comprise an AMP.

In some embodiments, a GLCS implements algorithms and medicament or glucose control functionality discussed herein to provide medicament or glucose control therapy without being connected to an AMD. For example, the GLCS can provide instructions or dose outputs that direct a user to administer medicament to provide glucose control therapy. In some implementations, the user may use, for example, a medicament pen to manually or self-administer the medicament according the GLCS's dose outputs.

In some implementations, the user may also provide inputs such as glucose level readings into the GLCS for the GLCS to provide dose outputs. The user inputs into the GLCS may be in combination with inputs from other systems or devices such as sensors as discussed herein. In some implementations, the GLCS can provide glucose control therapy based on user instructions without other system or device inputs.

In some embodiments, the GLCS includes a memory that stores specific computer-executable instructions for generating a dose recommendation and/or a dose control signal. The dose recommendation and/or the dose control signal can assist with glucose level control of a subject via medicament therapy. The dose recommendation or dose output of the GLCS can direct a user to administer medicament to provide medicament therapy for glucose level control, including manual administration of medicament doses. In additional embodiments, the GLCS includes the memory and a delivery device for delivering at least a portion of the medicament therapy. In further embodiments, the GLCS includes the memory, the delivery device, and a sensor configured to generate a glucose level signal. The GLCS can generate the dose recommendation and/or the dose control signal based at least in part on the glucose level signal.

In certain embodiments, the dose recommendation and/or the dose control signal can additionally be based at least in part on values of one or more control parameters. Control parameters can include subject-specific parameters, delivery device-specific parameters, glucose sensor-specific parameters, demographic parameters, physiological parameters, other parameters that can affect the glucose level of the subject, or any combination of one or more of the foregoing.

In some examples, the ambulatory medical device (AMD) is an electrical stimulation device, and therapy delivery includes providing electrical stimulation to a subject. An example of an electrical stimulation device is a cardiac pacemaker. A cardiac pacemaker generates electrical stimulation of the cardiac muscle to control heart rhythms. Another example of an electrical stimulation device is a deep brain stimulator to treat Parkinson's disease or movement disorders.

A method comprised of the instant teachings, as embodied in the figures and claims below makes the distinction at least a point of novelty. As discussed, and shown to those of skill in the art according to the Figures, the syringe inserted into the reservoir fills the same (with for example, insulin) and the plunger stays while the depicted nut turns with the lead screw until the nut passes a point where it is locked in with rotation of the system and the tooth engages the nut and stops it from locking. Again, once the syringe with insulin is filled the plungers stays then the nut turns with lead screw.

In combination with the other aspects of these inventions and systems, both disposable and durable versions are available targeted for three days, and the latter for at least about two years. Power management allows this system to be a non-rechargeable device along with the cannula insertion being made more precise according to BETAB24.0113Prov, namely the placement of the cannula being buffered and shielded allows for more user friendly application.

Likewise, the types of pumps include all known AMD and patch pumps, or any other related or unrelated systems design to move fluid from a reservoir (or the like means) into a patient, animal or other test-subject, laboratory scheme or the like schema. The devise shown are only provided in enough detail to explain and provide technical disclosure adequate to tech those skilled in the art the metes and bounds of the instant inventions. By way of example and for further explanation, discussion of a standard AIVID is offered for consideration as an illustrative but not limiting example.

The AMD can be connected to an infusion site using an infusion set. The AMD generally shall include a medicament pump and an integrated user interface that permit a user to view pump data and change therapy settings via user interaction with the user interface elements of the user interface. An analyte sensor, such as a glucose level sensor or a glucose sensor, generates a glucose level signal that is received by the glucose level control system. In some variants, the analyte sensor can include an insulin level sensor that can generate an insulin level signal that can be received by the glucose level control system. In some variants, the analyte senor can include a glucose level sensor and/or an insulin level sensor. In some variants, the analyte sensor may include a continuous glucose monitor (CGM).

The AMD (e.g., a medicament pump) includes an integrated cannula that inserts into the infusion site without a separate infusion set. At least some of the pump controls can be manipulated via user interaction with user interface elements of an external electronic device. In some instances, pump controls can be manipulated via user interaction with user interface elements generated by a remote computing environment (not shown), such as, for example, a cloud computing service, that connects to the AMD (medicament pump) via a direct or indirect electronic data connection.

By way merely of further examples, glucose level control systems typically include a user interface configured to provide one or more of therapy information, glucose level information, and/or therapy control elements capable of changing therapy settings via user interaction with interface controls. For example, the user can provide an indication of the amount of the manual bolus of medicament from an electronic device remote from the medicament pump. The user interface can be implemented via an electronic device that includes a display and one or more buttons, switches, dials, capacitive touch interfaces, or touchscreen interfaces, or voice interfaces. In some embodiments, at least a portion of the user interface is integrated with an ambulatory medicament pump that can be tethered to a body of a subject via an infusion set configured to facilitate subcutaneous injection of one or more glucose control agents. In certain embodiments, at least a portion of the user interface is implemented via an electronic device separate from the ambulatory medicament pump, such as a smartphone.

By way of example, the instant system works well with of a glucose level control system. As shown in the patents incorporated expressly by reference herein, a glucose level control system may comprise an ambulatory medical device (AMD) that includes a controller having an electronic processor and a memory that stores instructions executable by the electronic processor. In some cases, the pump can be an infusion pump for administering regulatory agent and/or counter-regulatory agent. and an insulin sensor.

A controller can be configured to generate the dose control signal using a control algorithm that generates at least one of a basal dose, a correction dose, and/or a meal dose (or food intake). Examples of some control algorithms that can be used to generate these doses are disclosed in U.S. Patent Application Publication Nos. 2008/0208113, 2013/0245547, 2016/0331898, and 2018/0220942 (referenced herein as the “Controller Disclosures”), or in the PCT Patent Application Publication No. W0 2021/067856, the entire contents of which are incorporated by reference herein and made a part of this specification. The correction dose can include regulatory or counter-regulatory agent and can be generated using a model-predictive control (M.C.) algorithm and/or other algorithms such as those disclosed in the Controller Disclosures. The basal dose can include regulatory agent and can be generated using a basal control algorithm such as disclosed in the Controller Disclosures. The meal dose can include regulatory agent and can be generated using a meal control algorithm such as disclosed in the Controller Disclosures. In some cases, a meal dose can be generated by the subject via a user interface of the glucose level control system for a subject without substantial user intervention while the controller remains in online mode. In some examples, the ambulatory medicament pump can include one or more medicament cartridges or can have an integrated reservoir of medicament, using a control scheme such as described in U.S. Pat. No. 7,806,854, the contents of which are hereby incorporated by reference in its entirety herein.

Pumps according to embodiments of the invention may operate several different ways, for example, in the offline mode, the controller may generate dose control signals as described in U.S. Pat. No. 10,543,313, the entire contents of which are hereby incorporated by reference in its entirety herein. In offline mode, the control algorithm generates a dose control signal that implements correction doses in response to isolated glucose measurements (such as, for example, measurements obtained from the subject using glucose test strips) and/or insulin measurements and based on control parameters of the control algorithm. The pump is configured to deliver basal doses to the subject without substantial user intervention and can deliver correction doses to the subject in response to isolated glucose measurements and/or isolated insulin measurements while the controller remains in offline mode.

As an alternative example, the control algorithm may include a linear algorithm that models diminishing glucose or the accumulation of glucose in the subject based on a linear reduction rate. For example, the control algorithm may determine that a particular dose, D, of insulin is to be administered to the subject. The control algorithm may then estimate that 0.25*D of the insulin is absorbed into the blood plasma per hour over 4 hours. Similarly, the control algorithm may estimate that the insulin diminishes at a rate of 0.33*D per hour over three hours upon the insulin reaching maximum concentration within the blood plasma.

Regardless of the control algorithm used, the automated glucose level control system may administer insulin and, in some cases, a counter-regulatory agent one or more times over a particular time period. There may be multiple reasons and/or triggers that cause the automated glucose level control system to supply insulin. For example, the automated glucose level control system may provide a basal does of insulin on a periodic basis in an attempt to maintain a steady glucose level in the blood plasma of the subject. As another example, the automated glucose level control system may supply mealtime boluses of insulin to account for an expected amount of glucose to be consumed as part of a meal. The mealtime bolus may be an amount specified by a user or may be an amount of insulin administered in response to an indication of meal size by the subject. This indication of meal size may be subjective. In some cases, the size of the bolus of insulin for an identified meal size may be a fixed or constant value.

In some other cases, the size of the bolus of insulin for an identified meal size may vary over time as the automated glucose level control system learns or refines the amount of insulin to administer to a subject to keep the subject's blood glucose within a target setpoint. The automated glucose level control system may learn or refine the optimal insulin to administer based on a comparison of expected glucose level measurements to actual glucose level measurements when the subject (or other user) makes a subjective identification of meal size. In addition to basal and mealtime boluses of insulin, the automated glucose level control system may also supply correction doses of insulin to the subject based on the glucose level signal. The correction doses of insulin may be supplied in response to a model predictive controller (M.C.) determining or estimating that a user's level of insulin is expected to fall below a threshold in some future period of time based on glucose level readings. The M.C. may execute a control algorithm that can regulate glucose concentration to a reference setpoint while simultaneously minimizing both the control signal aggressiveness and local insulin accumulation. A mathematical formulation describing the subcutaneous accumulation of administered insulin may be derived based on nominal temporal values pertaining to the pharmacokinetics of insulin in the subject. The mathematical formulation may be in terms of the insulin absorption rate, peak insulin absorption time, and/or overall time of action for the insulin (or another medicament).

Examples of an MPC controller that may be used with embodiments of the present disclosure are described in U.S. Pat. No. 7,806,854, issued on Oct. 5, 2010, the disclosure of which is hereby incorporated by reference in its entirety herein for all purposes.

The automated glucose level control system may track insulin therapy administered to the subject over a tracking period. Although the tracking period is not limited in length and may generally be any period of time, typically the tracking period is at least a minimum period of time sufficient for the automated glucose level control system to learn or refine the amount of medicament (e.g., insulin) to administer to the subject under particular conditions (e.g., when particular glucose levels are detected or when particular meal sizes are identified). For example, the automated glucose level control system may initially administer 6 units of insulin for lunch and 10 units of insulin for dinner. These initial values may be set be a healthcare provider and/or a subject based, for example, on clinical data for the subject.

However, over time (e.g., 3-5 days), the automated glucose level control system may determine that providing 7 units of insulin for lunch and 8 units of insulin for dinner maintains the subject's glucose level closer to the median of the setpoint range than did the initial configuration. Although not limited as such, generally each unit of insulin is 1/100th of a milliliter of insulin. As indicated, the tracking period can be any length of time. For example, the tracking period could be 1 day, 3 days, 5 days, 7 days, anything in between, or more. Typically, the tracking period is at least long enough to provide sufficient time to learn or refine initial settings of the automated glucose level control system for the subject. In some cases, the tracking period may be 1 or 2 days. In other cases, the tracking period may be from a particular time period until a current time period. For example, the tracking period may be from the start of therapy until a current point in time. In other cases, the tracking period may be a moving or shifting window. For example, the tracking period may be the least week, two weeks, month, or year. Further, for non-blood glucose systems, the tracking period may differ based on the amount of time sufficient to determine or refine medicament control values for the subject. In some cases, the tracking period may a window of a particular length. This window may be a moving window. For example, the window may be the previous 7 days. As time passes, the window moves to continue to encompass the previous 7 days.

Tracking the insulin therapy may include storing the autonomously determined doses of insulin delivered to the subject. These autonomously determined doses of insulin may include one or more of basal insulin doses, mealtime insulin boluses, or correction insulin doses. Moreover, tracking the insulin therapy may including tracking the type of insulin used. The type of insulin may include any type of insulin, such as fast-acting insulin (e.g., Lispro, Aspro, or Glulisin), regular or short-acting insulin (e.g., Ilumulin R, Novolin R, or Velosulin R), intermediate-acting insulin (e.g., Humulin N, Novolin N, ReliOn), long-acting insulin (e.g., detemir (Levemir), and glargine (Basaglar, Lantus)), or Ultra long-acting insulin (e.g., degludec (Tresiba), glargine u-300 (Toujeo)). Further, tracking the insulin therapy may include tracking counter-regulatory agent (e.g., Glucagon) therapy.

In some cases, tracking the insulin therapy may include calculating average therapy provided over a period of time (e.g., over the tracking window). For example, the tracking of the insulin therapy may include determining a moving average of the past 7 days of nominal basal doses during each dosing interval. Assuming basal therapy is provided every five minutes, the moving average may be calculated based on the previous 288 doses (e.g., over 1 day) or 2016 doses (e.g., over 7 days). This calculation can be used to obtain a basal rate profile for backup therapy. In some cases, the time period may be broken up into different time segments that may be associated with different rates of therapy. For example, there may be 4 basal therapy periods (e. g., 10 pm-4 am, 4 am-10 am, 10 am-4 pm, and 4 pm-10 pm). Thus, a separate moving average may be calculated for each of the basal therapy periods over a day, or over some other time period (e. g., 7 days). The calculated averages may be used to calculate a backup basal rate that can be used to program an automated glucose level control system. Further, the basal rate profile may include aggregating the doses across the day to determine a dose of long-acting insulin that can be used for injection therapy.

Similar to the basal therapy, a moving average of correction doses can be calculated to determine a correction bolus of insulin to supply via a pump or injection therapy. Alternatively, or in addition, the moving average of correction doses in combination with measurements of blood glucose of the subject over time may be used to determine a rate of change of blood glucose from a unit of insulin provided during correction therapy.

Mealtime boluses may also be calculated using a moving average. Further, a separate moving average may be calculated for each meal (e. g., breakfast, lunch, and dinner) dose over some period of time (e.g., 7 previous days of mealtimes). In some cases, each of the moving averages may be calculated using different windowing functions. For example, the moving average may be calculated using a Hann window or a Hamming window. In some cases, different levels of dosing may be determined for different meal sizes and different doses may be determined for different meals. In some cases, different meals (e.g., breakfast vs lunch) may have different dosing despite similarity in size due, for example, to differences in the subject's glucose levels when they wake up versus when they usually have lunch, or because differences in types of foods consumed at breakfast versus lunch. Further, in some cases, differences in metabolisms of different subjects may result in different mealtime boluses.

The insulin therapy may be stored in a therapy log, or any other type of data structure. Further, the insulin therapy may be stored in a memory of the automated blood glucose system, on a companion device, on a computing device of the subject or user (e.g., a laptop or desktop), in a cloud computing environment, or in any other storage system capable of receiving the insulin therapy information from the automated glucose level control system. Using the therapy log or tracked insulin data, the automated blood glucose system, or a computing system With access to the therapy log or tracked insulin data, may generate a backup insulin therapy protocol. The backup insulin therapy protocol may include a backup injection therapy protocol or a backup pump therapy protocol. The backup injection therapy protocol may include one or more amounts of insulin (or other medicament) to administer using injection therapy (e.g., manually provided shots) at one or more times to help maintain the subject's condition within a normal or desired physiological range or condition. The backup pump therapy protocol may include data and/or instructions for a replacement medicament pump of the same type or of a different type to supply therapy to the subject. The replacement medicament pump may be a permanent replacement or a temporary replacement. The backup pump therapy protocol may be the same as and/or include the same type of information as the backup injection therapy protocol. Alternatively, or in addition, the backup pump therapy protocol may include different values than the backup injection therapy protocol. For example, the backup pump therapy protocol may include an indication of basal therapy to provide periodically on relatively short increments (e.g., every 5 minutes, every half hour, every hour, etc.) Because an insulin pump may automatically administer insulin, it is possible to provide a steady or periodic drip of insulin. It may be impractical for a subject using injection therapy to administer insulin manually on similar short increments. Instead, a user might administer therapy on a less regular basis (e.g., once every roughly 4-5 hours or 6-8 hours, prior to mealtimes, after waking, and/or before sleeping, etc.). Accordingly, the backup therapy protocol for a pump and for injection may differ. Further, the type of insulin used or identified in the backup protocol may differ. For example, the backup protocol may call for use of long-acting insulin, such as, for example, insulin glargine, or intermediate-acting insulin, such as, for example human recombinant insulin.

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein. All of the processes described herein may be embodied in, and fully automated via, software code modules executed by a computing system that includes one or more computers or processors. The code modules may be stored in any type of non-transitory computer-readable medium or other computer storage device. Some or all the methods may be embodied in specialized computer hardware. Further, the computing system may include, be implemented as part of, or communicate with an automated blood glucose system, an ambulatory medicament system, or an ambulatory medical device.

A particularly advantageous aspect of the invention is the provision of a durable patch module and a disposable patch module connectable to one another to aggregately form a coupled patch pump assembly for active use.

### BRIEF DESCRIPTION OF THE FIGURES

FIG. 1 is a perspective view of an embodiment of a patch pump assembly according to the invention viewed in a direction of an outward facing side thereof having therein a pump system effective for using a drive mechanism to deliver fluid from a reservoir into a recipient;

FIG. 2 is a perspective view of the patch pump assembly depicted in FIG. 1, viewed in a direction of a working contact surface including depiction of a cannula used for fluid delivery;

FIG. 3 is a detailed view of workings of the embodiment of FIGS. 1 and 2 with barriers to view an interior schematically removed;

FIG. 4 is another detailed view of the embodiment shown in plan of workings of the instant system with barriers to view schematically removed;

FIG. 5 is a further detailed view of workings of the instant system with additional barriers to view schematically removed;

FIG. 6 illustrates a balance of the instant system with barriers to view schematically removed;

FIG. 7 depicts a balance of the instant system with barriers to view schematically removed;

FIG. 8 illustrates a balance of the instant system with barriers to view schematically removed;

FIG. 9A depicts an explanatory view of a durable module and a disposable module in a process of engageable coupling one to the other;

FIG. 9B is a explanatory view showing a completed assembly comprising the durable module coupled to the disposable module;

FIG. 10 is a schematic representation of an embodiment of the invention showing at least a portion of operational components included in an interior of a patch pump assembly; and

FIG. 11 is a cross-section view taken at line 11-11 of FIG. 10, depicting inter alia a gear set for transmitting rotation from a drive motor to a pump portion, advantageously provided in the durable module to operative components in the disposable module.

### DETAILED DESCRIPTION OF THE INVENTION

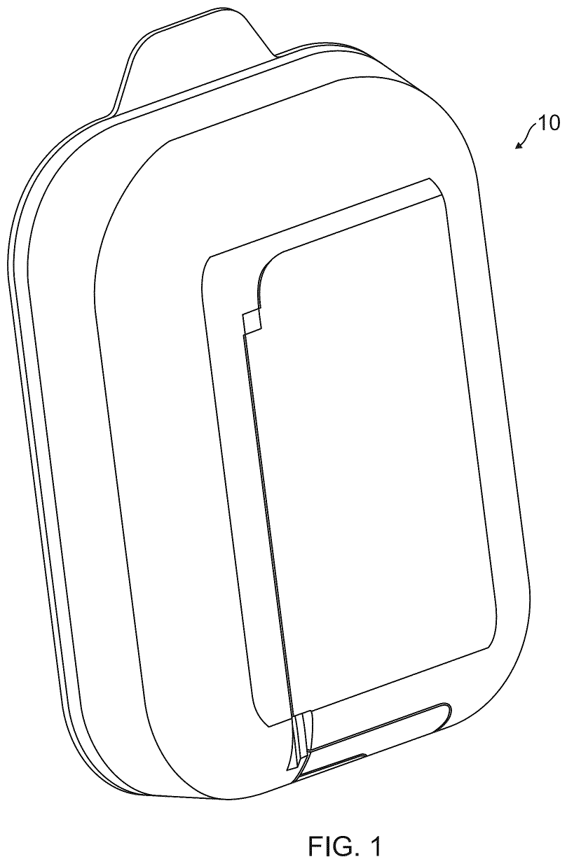

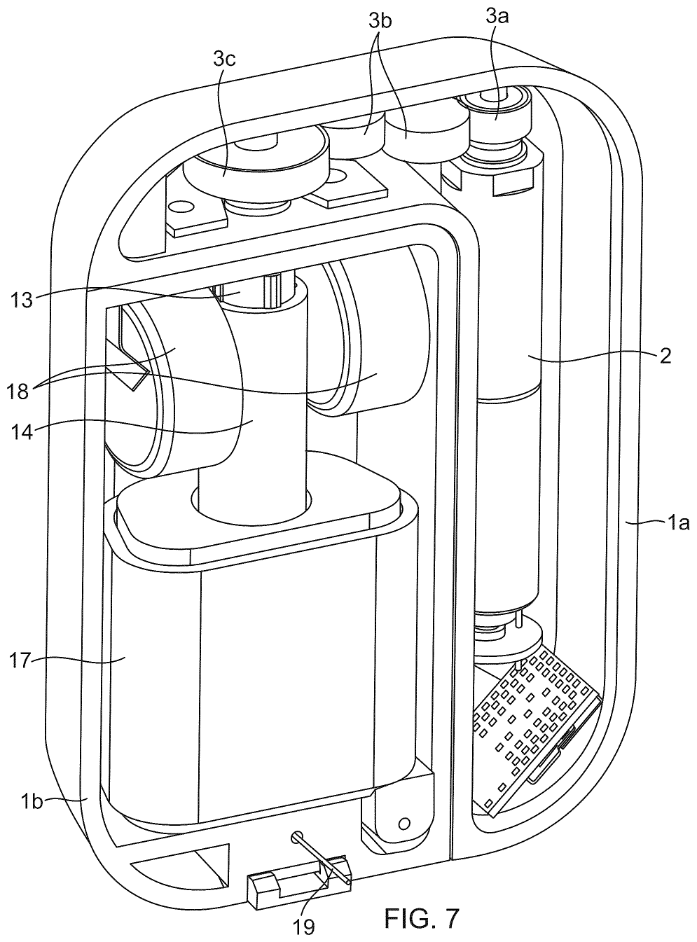

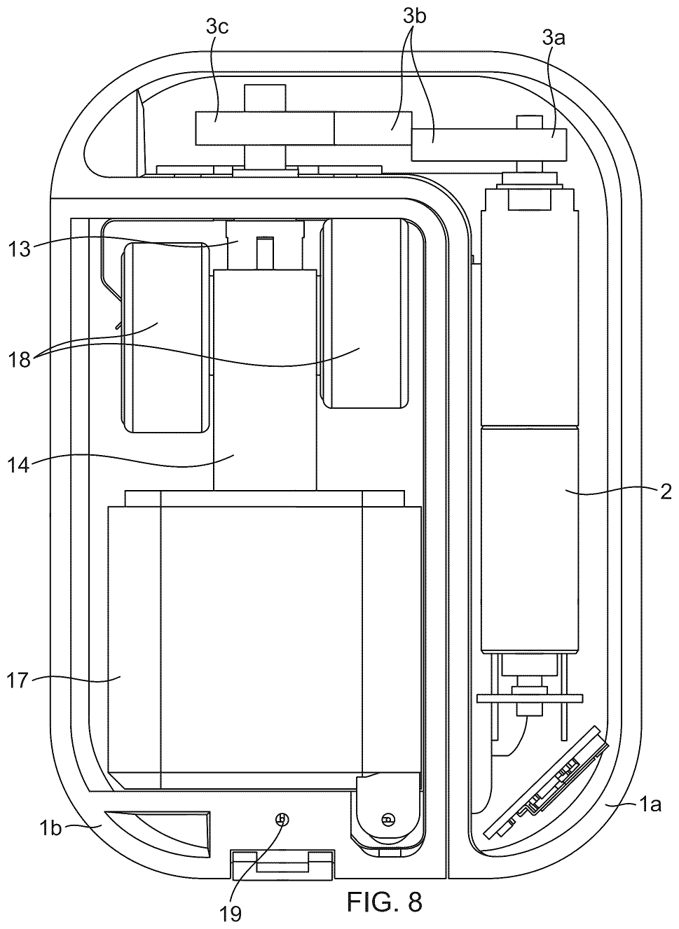

FIGS. 1-8 show examples of an ambulatory medical device (AMD), such as an ambulatory medicament pump (AMP), connectable to a subject. Those skilled in the art will understand that those improvements denoted herein are applicable across the board to extant systems, such as those of BETA BIONICS, INC (Concord, Mass) along with those of Medtronic Minimed; Tandem Diabetes Corp.; Insulet Corp.; EO Flow; Inreda, BV and other medical device providers. Reference is made likewise to the Appendix offering for consideration state of the art and prior art versions of means for pushing fluids.

Referring now to FIG. 1, a partial perspective dorsal view represents conventional pump dynamics and those skilled in the art likewise are well aware that is interchangeable with known iterations of patch pumps. Gross surface morphology is illustrative rather than limiting and shows the orientations of the respective improvements offered for consideration herein, as discussed the enumerated improvements to drive coupling and fill detection schemas shall become known to those of skill in the art from these figures.

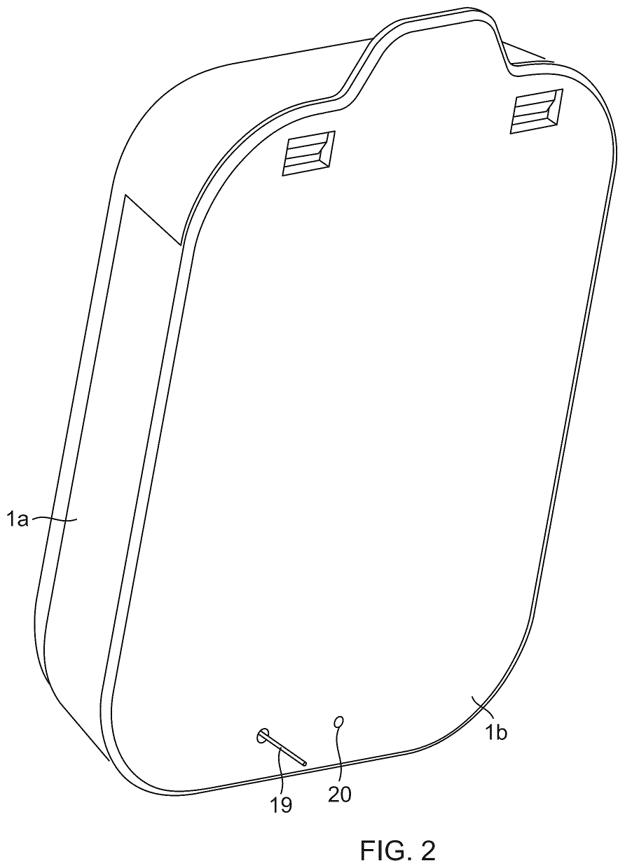

Referring now also to FIG. 2, the ventral view depicts a not-scaled yet partial perspective view of the proposed newly innovated housing for the revised and improved inner workings, namely for delivering fluid delivery profiles for priming, bolus delivery and continuous and variable rate delivery. Cannula with incumbent introducer/trocar or having adequate stiffness is shown at the bottom of the figure, which those of skill in the art are readily able to understand to be an endpoint for the fluid delivery path which begins with the subject reservoir, as shown in the remaining figures, it is respectfully submitted.

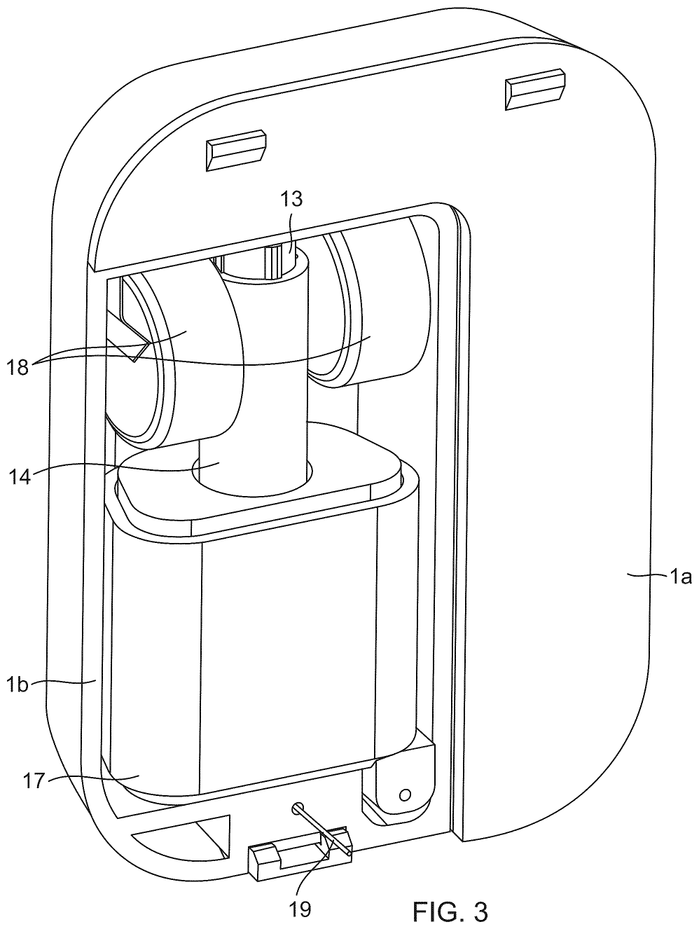

Now referring also to FIG. 3, the perspective view is maintained along with the inner working being further revealed for clarity not comprehensive viewing. Namely, the mechanical relationship between the cannula and other end of the fluid path is depicted, with an immediate view of an improvement in that no “clutch-type” of mechanism is needed because the syringe inserted into the reservoir fills the same (with for example, insulin) and the plunger stays while the depicted nut turns with the lead screw until the nut passes a predetermined interval where it is locked in with rotation of the system and the tooth engages the nut and stops it from locking.

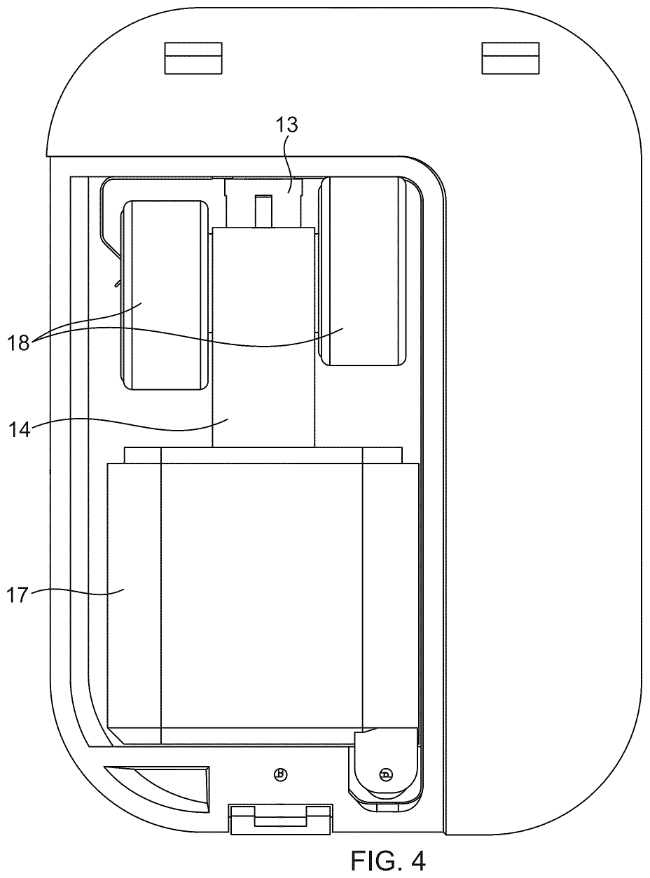

Referring to FIG. 4, the plunger stays in position and an optional trigger switch (not shown) is, for example, a contact switch, causing travel to the interface to determine insulin (or other medicament fill.

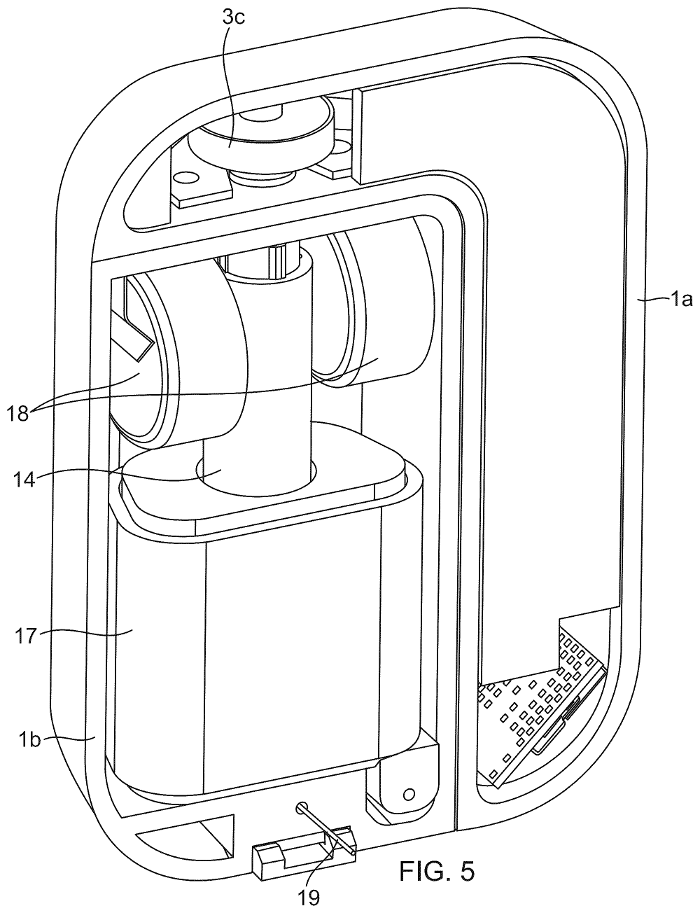

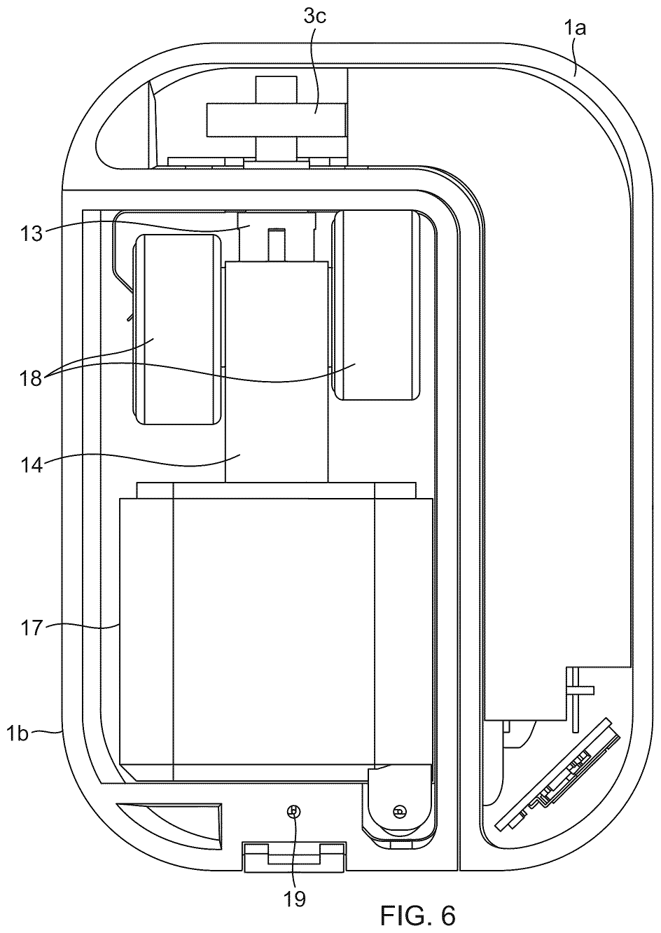

Referring now also to FIGS. 5-8, the bottom right (as rotated) shows the circuit board also housing the audio and alarm circuitry, completing the drive coupling and sensing method for fill detection, which is amenable to treatment with various power options, namely both non-rechargeable and battery systems. It is noted that disposable (for example 3 days) and durable (up to 2 years) versions are available according to the instant teachings. The illustrated one-way drive coupling clearly is differentiated from the prior art (see for example the appendix including TNSULET U.S. Pat. Nos. 11,22,741; 10,420,993 and 9,402,950—along with excerpts from a Fed. District Court providing a claim construal supporting this statement with respect to the same patents). The one-way drive coupling and fill detection by trigger/contact switch facilitates travel to the interface and determines the insulin fill, with respect to FIGS. 6-8, unlike the differentiated prior art, it is respectfully submitted. Likewise disclosed according to the instant teachings is the method of operating the instant fluid delivery devices, once again, teaching away is the prior art which requires “a clutch mechanism coupled to the drive wheel” by being a central feature, and the focus of half of a dozen independent claims.

In FIGS. 1-8, the glucose level control system includes for example the AMD (e.g., a medicament pump) which communicates with an external electronic device (such as, for example, a smartphone) via a wireless data connection. At least some of the pump controls can be manipulated via user interaction with user interface elements in the user interface of the external electronic device. The glucose level sensor can also communicate with the AMD (that includes a medicament pump) via a wireless data connection. Example user interfaces that can be implemented by one or more of the external electronic device, the AMD, a remote electronic device (not shown), and/or other electronic devices are shown and described in PCT Patent Application Publication Nos. WO 2021/067767 and WO 2021/011699, the entire contents of which are hereby incorporated by reference herein and made a part of this specification.

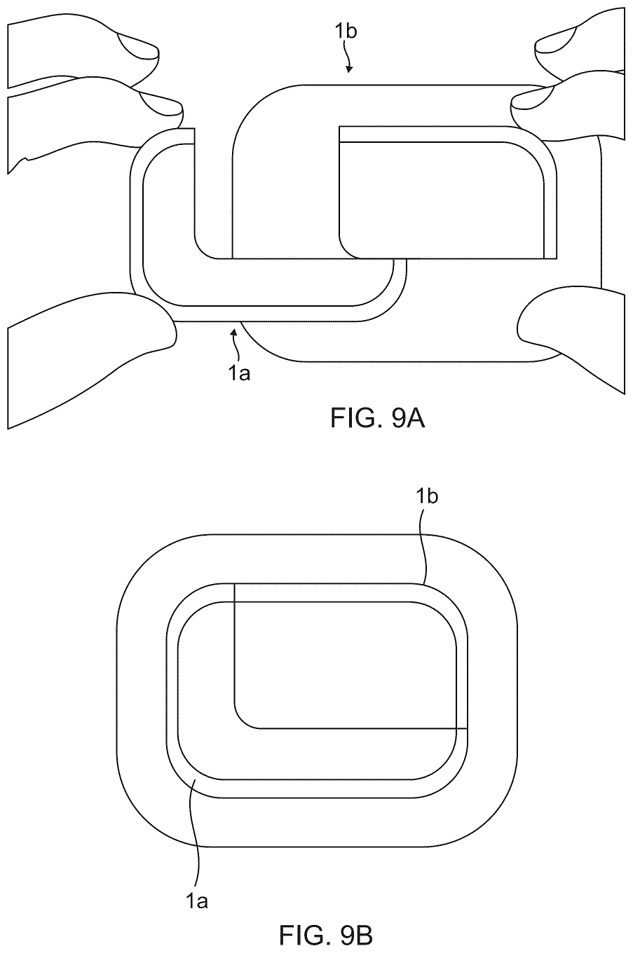

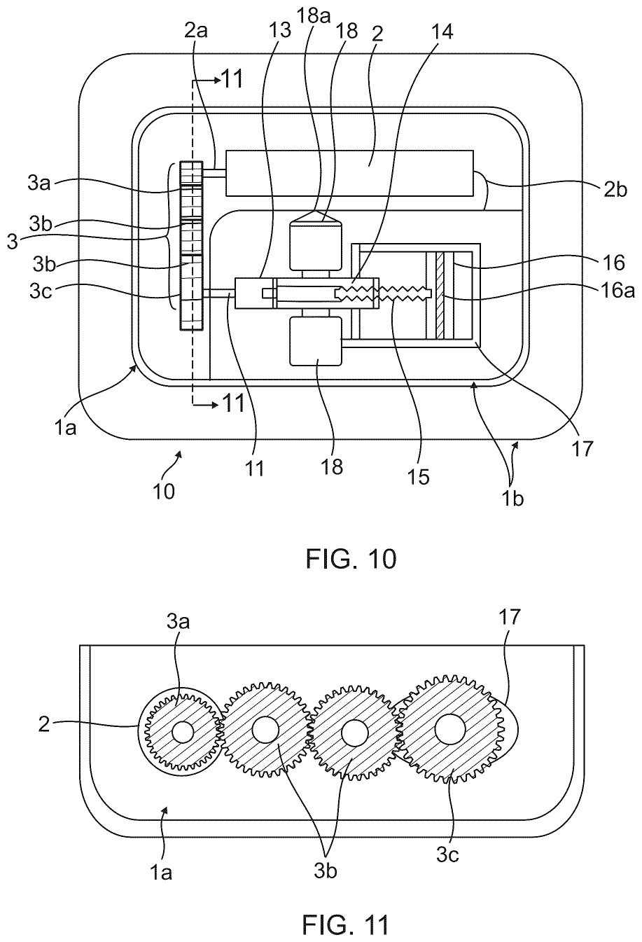

Referring now to FIGS. 9A and 9B, a particularly advantageous embodiment is depicted, and which illustrates a patch assembly 10 (see FIG. 1) comprised of a durable patch module 1a and a disposable patch module 1b (e.g., in a form of a blister pack), configured for mutually engaged connection one to another. As depicted schematically in FIG. 9A, durable patch module la is slid relative to disposable patch module 1b in order to achieve a coupled aggregate unit comprising patch assembly 10 as shown in FIG. 9B. As will be described in further detail with reference to FIG. 10, the provision of durable patch module la and disposable patch module 1b provides particular advantages.

Turning now to FIG. 10, patch assembly 10 includes durable patch module 1a and disposable patch module 1b. In advantageous manner, an enclosed interior of durable patch module 1a includes durable components which can operate for extended periods without requiring replacement, and parts which are isolated from direct contact with a patient. An interior of disposable patch module 1b conversely houses operational elements that require periodic replenishment, as will be described with reference to the schematically depicted example of FIG. 10.

As shown in the illustrated example of FIG. 10, durable patch module 1a comprises a drive motor 2 actuatable to create rotational forces that can be transferred to elements within disposable patch module 1b, via, inter alia, output shaft 2a, when durable patch module 1a is brought into coupled engagement therewith. A housing of disposable patch module 1b has a longitudinal extent crosswise to a coupling end. Durable patch module 1a is advantageously configured to have a form complementary to that of disposable patch module 1b to permit reliable mutual interconnection. For example, durable patch module 1a can be embodied in a form including a mechanically operable portion capable of physically transferring rotation produced by drive motor 2 to components within disposable patch module 1b by being brought into general alignment with the coupling end of disposable patch module 1b, and an arm portion extending generally parallel to the longitudinal extent of disposable patch module 1b and arranged roughly orthogonal to the operable portion, such that the operable portion is alienable with the coupling end of disposable patch module 1b, while a portion of components within durable patch module 1a is disposed adjacent to those of disposable patch module 1b, including drive motor 2.

A gear set 3 is provided at the operable portion of durable patch module 1a and is comprised for example of a pinion gear 3a corresponding to drive motor 2, a drive gear 3c, and a pair of idler gears disposed therebetween. Gear set 3 operates to transmit rotation from drive motor 2 to components within disposable patch module 1b.

Components within disposable patch module 1b advantageously include a drive coupling 11, a drive wheel 13 connected to drive coupling 11, a leadscrew 15, a leadscrew advancement element (depicted conveniently in a form of a leadscrew nut 14) being threadably engageable with leadscrew 15, and a plunger 16 sealed against an interior wall of a reservoir 17 conveniently by an o-ring 16a. Reservoir 17 and piston 16 are of a non-round configuration, for example having an elliptical cross-section, rendering leadscrew 15 non-rotational. As leadscrew nut 14 is rotated by drive wheel 13, leadscrew 15 and piston 16 are movable in two direction along a reservoir axis depending on a direction of rotation.

Durable patch module 1a optionally houses electronic circuitry (not identified by reference numeral(s) but regardless will be readily understood by a skilled artisan), and serves to control operation of patch assembly 10 in a manner assuring delivery of a proper dosage of the medicament contained in reservoir 17 of disposable patch module 1a. Alternatively, control of functions of patch assembly 10 can be done remotely from any suitable computing device (i.e., a cell phone or dedicated device) communicating through any acceptable protocol.

A battery 18 is provided in disposable patch module 1b which, when durable patch module 1a is coupled to disposable patch module 1b, serves to energize all powered components in durable patch module 1a via, for example, a power cable 2b.

As depicted in the various views of the figures, disposable patch module includes a central region housing operational components, and advantageously also a generally flattened flange at a periphery thereof to increase a surface area of a recipient contact surface on a side of disposable patch module 1b opposite to the central region housing. Conveniently, contact adhesive is present of the recipient contact surface, advantageously protected by release layer(s) until ready for affixing to a reception surface of the recipient, e.g., a patient, providing a means for attachment of patch assembly 10 to the patient.

A cannula 19 communicative with the output of reservoir 17 provides subcutaneous delivery of the medicament, for example insulin, to a patient when patch assembly is applied to the selected body surface.

FIG. 11 is a cross-sectional view taken along line 11-11 in FIG. 10, and shows the relative placements of gear set 3, including gears 3a, 3b and 3c, durable patch module.

Many other variations than those described herein will be apparent from this disclosure. For example, depending on the embodiment, certain acts, events, or functions of any of the algorithms described herein can be performed in a different sequence, can be added, merged, or left out altogether (for example, not all described acts or events are necessary for the practice of the algorithms). Moreover, in certain embodiments, acts or events can be performed concurrently, for example, through multi-threaded processing, interrupt processing, or multiple processors or processor cores or on other parallel architectures, rather than sequentially. In addition, different tasks or processes can be performed by different machines and/or computing systems that can function together.

The various illustrative logical blocks and modules described in connection with the embodiments disclosed herein can be implemented or performed by a machine, such as a processing unit or processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can include electrical circuitry configured to process computer-executable instructions. In another embodiment, a processor includes an PGA or other programmable device that performs logic operations without processing computer-executable instructions. A processor can also be implemented as a combination of computing devices, for example, a combination of a DSP and a microprocessor, a plurality of microprocessors, one or more microprocessors in conjunction with a DSP core, or any other such configuration. Although described herein primarily with respect to digital technology, a processor may also include primarily analog components. A computing environment can include any type of computer system, including, but not limited to, a computer system based on a microprocessor, a mainframe computer, a digital signal processor, a portable computing device, a device controller, or a computational engine within an appliance, to name a few. Conditional language such as, among others, “can,” “could,” “might” or “may,” unless specifically stated otherwise, are otherwise understood within the context as used in general to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, Whether these features, elements and/or steps are included or are to be performed in any particular embodiment.

Disjunctive language such as the phrase “at least one of X, Y, or Z,” unless specifically stated otherwise, is otherwise understood with the context as used in general to present that an item, term, etc., may be either X, Y, or Z, or any combination thereof (for example, X, Y, and/or Z). Thus, such disjunctive language is not generally intended to, and should not, imply that certain embodiments require at least one of X, at least one of Y, or at least one of Z to each be present.

Any process descriptions, elements or blocks in the ?ow diagrams described herein and/or depicted in the attached figures should be understood as potentially representing modules, segments, or portions of code which include one or more executable instructions for implementing specific logical functions or elements in the process. Alternate implementations are included Within the scope of the embodiments described herein in which elements or functions may be deleted, executed out of order from that shown, or discussed, including substantially concurrently or in reverse order, depending on the functionality involved as would be understood by those skilled in the art.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation working in conjunction with a second processor configured to carry out recitations B and C.

It should be emphasized that many variations and medications may be made to the embodiments described herein, the elements of which are to be understood as being among other acceptable examples. All such medications and variations are intended to be included herein within the scope of this disclosure.

What is claimed is:

1. An ambulatory patch device for administering at least one therapeutic treatment to a recipient, comprising:
components operable to facilitate a carrying out of the at least one therapeutic treatment;
a patch assembly which comprises a disposable patch module including at least a portion of said components, said patch assembly further comprising a durable module receivable to said disposable patch module, said durable patch module including at least a portion of a remainder of said components;
said at least a portion of said components associated with said disposable patch module being selected based on a determination of limited reusability; and
said at least a remaining portion of said components associated with said durable patch module including at least one characteristic deemed suitable for repeated use, said at least a portion of said components and said at least a remaining portion of said components working together to facilitate the carrying out of the at least one therapeutic treatment.

2. An ambulatory patch device according to claim 1, further comprising:
a leadscrew in threaded engagement with a leadscrew advancement element, said leadscrew being affixed to a plunger; and
a mechanism operable to impart rotation to said leadscrew advancement element including a gear set comprised of at least two intermeshed gears.

3. An ambulatory patch device according to claim 2, further comprising:
a reservoir which accommodates said plunger; and
said piston and said reservoir include a non-round cross-section rendering said leadscrew non-rotational.

4. An ambulatory patch device according to claim 3, further comprising a gear set comprised of at least two intermeshed gears.

5. An ambulatory patch device according to claim 1, wherein said therapeutic treatment includes delivering measured dosages of a fluid medicament to the recipient.

6. An ambulatory patch device according to claim 5, wherein said fluid medicament includes insulin.

7. An ambulatory patch device according to claim 5, wherein said components include at least one of a drive motor or a delivery pump.

8. An ambulatory patch device according to claim 5, wherein said durable patch module includes at least one of a drive motor or a gear set.

9. A pump device for pushing a metered quantity of a stored volume of a fluid medicament into a patient, comprising:
a drive motor including a rotational output rotating about a motor axis when energized;
a piston;
a reservoir including a reservoir axis having an interior space in which the volume of the medicament is receivable and an outlet through which the metered quantity is expellable, said piston being receivable within said reservoir moveable within the interior space along said reservoir axis so as to displace the metered quantity when moved in a direction of said outlet, said reservoir being located laterally adjacent to said drive motor, said reservoir axis and said motor axis running codirectionally;
a leadscrew which is rendered non-rotatable being affixed to said piston;
a leadscrew advancement element being threadably engageable with said leadscrew and substantially fixed with respect to movement along said reservoir axis, rotation of said leadscrew advancement element imparting movement of said piston along said reservoir axis; and
a mechanism operable to impart rotation to said leadscrew advancement element created by said drive motor.

10. A pump device according to claim 9, wherein said leadscrew includes outwardly facing threads and said leadscrew advancement element is comprised of a nut having internally facing threads.

11. A pump device according to claim 9, wherein said leadscrew includes internal threads and said leadscrew advancement element is comprised of outwardly facing threads.

12. A pump device according to claim 9, wherein said piston and said reservoir include a non-round cross-section rendering said leadscrew non-rotational.

13. A pump device according to claim 9, wherein said medicament includes insulin.

14. A pump device according to claim 9, wherein said mechanism operable to impart rotation to said leadscrew advancement element includes a gear set comprised of at least two intermeshed gears.

15. A pump device according to claim 9, further comprising:
a durable pump module and a disposable pump module configured for mutually engaged connection one to another so as to comprise a pump assembly;
components operable to facilitate a delivery of said medicament; and
at least one of said durable pump module or said disposable pump module including a housing within which at least a portion of said components are receivable.

16. A method of delivering a metered amount of a medicament subcutaneously to a patient; comprising;
selectively imparting rotation to a drive part threadably engaged with a non-rotatable lead screw affixed to a piston which is sealably movable along a reservoir axis of a reservoir when received therein;
threadably engaging the lead screw with a rotational part operable to impart movement of the piston along the reservoir axis when rotated relative to the leadscrew;
creating rotational movement by energizing a motor about a rotational axis; and
transmitting said rotational movement to a drive mechanism including the drive part.

17. A method according to claim 16, wherein said medicament includes insulin.

18. A method according to claim 16, wherein said transmitting includes using a gear set.

19. A method according to claim 16, wherein said non-rotatable lead screw affixed to said piston is rendered non-rotational at least in part by a non-circular cross-section of said piston and said reservoir.

20. A method according to claim 16, wherein said selectively imparting rotation being transmitted from an output of a drive motor.
